(19) United States
(12) Patent Application Publication
(10) Pub. No.: US 2013/0129841 A1
(43) Pub. Date: May 23, 2013

(54) THERAPEUTIC COMBINATION COMPRISING A PARP-1 INHIBITOR AND AN ANTI-NEOPLASTIC AGENT

(71) Applicants: Antonella Ciavolella, Bizzarone (CO) (IT); Alessia Montagnoli, Milan (IT); Enrico Pesenti, Parabiago (MI) (IT)

(72) Inventors: Antonella Ciavolella, Bizzarone (CO) (IT); Alessia Montagnoli, Milan (IT); Enrico Pesenti, Parabiago (MI) (IT)

(73) Assignee: NERVIANO MEDICAL SCIENCES S.r.l., Nerviano (MI) (IT)

(21) Appl. No.: 13/813,321

(22) PCT Filed: Jul. 25, 2011
(86) PCT No.: PCT/EP2011/062759

§ 371 Date: Jan. 30, 2013

(30) Foreign Application Priority Data
Aug. 3, 2010 (EP) 10171756.9

(51) Int. Cl. A61K 31/454; A61K 31/7068; A61K 45/00; A61K 31/4545; A61K 31/4188; A61K 31/337; A61K 33/24; A61K 31/5377

(52) U.S. Cl. CPC A61K 31/454; A61K 33/24; A61K 31/7068; A61K 31/5377; A61K 31/4545; A61K 31/4188; A61K 31/337; A61K 45/00
USPC 424/649; 514/49; 514/231.5; 514/316; 514/323; 514/393; 514/449

(57) ABSTRACT

The present invention provides a therapeutic combination comprising (a) a compound of formula (I) as set forth in the specification and (b) one or more antineoplastic agents selected from the group consisting of an alkylating or alkylating-like agent, an antimetabolite agent, a topoisomerase I inhibitor, a topoisomerase II inhibitor, an antimitotic agent and radiation wherein the active ingredients are present in each case in free form or in the form of a pharmaceutically acceptable salt or any hydrate thereof.

The present invention relates in general to the field of cancer treatment and, more particularly, provides an anti-tumor composition comprising a selected group of PARP-1 inhibitors and one or more antineoplastic agents selected from the group consisting of an alkylating or alkylating-like agent, an antimetabolite agent, a topoisomerase I inhibitor, a topoisomerase II inhibitor, an antimitotic agent and radiation.

### BACKGROUND ART

Poly (ADP-ribose) polymerases belong to a family of 18 members that catalyze the addition of ADP-ribose units to DNA or different acceptor proteins, which affect cellular processes as diverse as replication, transcription, differentiation, gene regulation, protein degradation and spindle maintenance. PARP-1 and PARP-2 are the only enzymes among the PARPs that are activated by DNA damage and are involved in DNA repair.

PARP-1 is a nuclear protein consisting of three domains: the N-terminal DNA-binding domain containing two zinc fingers, the auto modification domain, and the C-terminal catalytic domain. PARP-1 binds through the zinc-finger domain to DNA single strand breaks (SSB), cleaves NAD+, and attaches multiple ADP-ribose units to target proteins such as histones and various DNA repair enzymes. This results in a highly negatively charged target, which in turn leads to the unwinding and repair of the damaged DNA through the base excision repair pathway. In knock out mouse models, deletion of PARP-1 impairs DNA repair but it is not embryonic lethal. Double knock out PARP-1 and PARP-2 mice instead die during early embryogenesis, suggesting that the two enzymes display not completely overlapping functions. Enhanced PARP-1 expression and/or activity have been shown in different tumor cell lines, including malignant lymphomas, hepatocellular carcinoma, cervical carcinoma, colorectal carcinoma, leukemia. This may allow tumor cells to withstand genotoxic stress and increase their resistance to DNA-damaging agents. As a consequence, inhibition of PARP-1 through small molecules has been shown to sensitize tumor cells to cytotoxic therapy (e.g. temozolomide, platinums, topoisomerase inhibitors and radiation). A significant window seems to exist between the ability of a PARP inhibitor to potentiate therapeutic benefits and undesirable side effects. Whereas the therapeutic use of PARP inhibitors in combination with DNA damaging agents is not novel, the use of these agents as monotherapy, in particular tumor genetic backgrounds deficient in the homologous recombination DNA repair, represents a new approach. Individuals with heterozygous germ line mutations in either the BRCA-1 or BRCA-2 homologous recombination repair genes exhibit high life time risks of developing breast and other cancers. Tumors arising in mutation carriers have generally lost the wild type allele and do not express functional BRCA-1 and BRCA-2 proteins.

Therefore, loss of these two proteins leads to a tumor-specific dysfunction in the repair of double strand breaks by homologous recombination. It is known that when PARP-1 is inhibited, base excision repair is reduced and single strand breaks that are generated during the normal cell cycle persist. It has also been established that replication forks that encounter an unrepaired break can form double strand breaks which are normally repaired by homologous recombination. Tumor cells that are deficient in homologous recombination repair such as BRCA-1 and BRCA-2 mutants are therefore highly sensitive to PARP inhibition compared with wild-type cells. This is in line with the concept of synthetic lethality, in which the two pathway defects alone are innocuous but combined become lethal: PARP inhibitors may be more effective in patients with tumors with specific DNA repair defects without affecting normal heterozygous tissues. Putative patient population includes, besides BRCA mutants that represent the majority of hereditary breast and ovarian cancer, also a substantial fraction of sporadic cancers with defects in homologous recombination repair, a phenomenon termed “BRCAness”. For example, methylation of the promoters of the BRCA-1 or FANCF genes and amplification of the EMSY gene, which encodes a BRCA-2 interacting protein. By extending the rational of synthetic lethality of PARP and BRCA-1 and BRCA-2, it is likely that deficiencies in any gene that is not redundant in double strand break repair should be sensitive to PARP inhibition. For example, ATM deficiency, found in patients with T-cell prolymhocytic leukemia and B-cell chronic lymphocitic leukemia and breast cancer, and CHK2 germ line mutations identified in sarcoma, breast cancer, ovarian cancer and brain tumors, have also been shown to be synthetically lethal in combination with PARP deficiency as well as deficiencies in other known HR pathway proteins (including RAD51, DSS1, RAD54, RPA1, NBS1, ATR, CHK1, CHK2, FANCD2, FANCA, and FANCC). pTEN mutations are also considered a genetic background synthetically sensitive to PARP inhibition. The first clinical evidence that BRCA-mutated cancer may be sensitive to PARP inhibitor monotherapy comes from the preliminary data for the phase I trial of the oral, small molecule PARP inhibitor, AZD2281. In an enriched phase I population for BRCA mutation carriers, partial responses were seen in 4 out of 10 ovarian cancer patients with confirmed BRCA-1 mutations. Other PARP inhibitors, such as AG014699, BSI-201, are currently known to be in phase II and phase III clinical trials both in combination with DNA damaging agents and as single agent in BRCA deficient tumors. Early indications are that these therapies show low toxicity. Anyway compounds with high selectivity on PARP-1 are expected to show even less toxicity in view of a chronic treatment schedule.

PARP-1 has also been implicated in angiogenesis. In particular, PARP-1 inhibition seems to result in decreased accumulation of the transcription hypoxia-inducible factor la an important regulator of tumor cell adaptation to hypoxia.

Pro-inflammatory stimuli trigger the release of pro-inflammatory mediators that induce the production of peroxynitrate and hydroxyl radicals, which in turn yield to DNA single strand breakage with consequent activation of PARP-1. Over activation of PARP-1 results in depletion of NAD+ and energy stores, culminating in cell dysfunction and necrosis. This cellular suicide mechanism has been implicated in the pathomechanism of stroke, myocardial ischemia, diabetes, diabetes-associated cardiovascular dysfunction, shock, traumatic central nervous system injury, arthritis, colitis, allergic encephalomyelitis and various other forms of inflammation. Of special interest is the enhancement by PARP-1 of nuclear factor kB-mediated transcription, which plays a central role in the expression of inflammatory cytokines, chemokines and inflammatory mediators. The co-pending patent application PCT/EP2010/059607 in the name of the present Applicant, describes certain 3-oxo-2,3-dihydro-1H-isoindole-4-carboxamides having PARP-1 inhibitory activity.

Drugs that target DNA replication elongation are widely used in chemotherapy, for example, gemcitabine, active metabolites of 5-fluorouracil and hydroxyurea, topoisomerase inhibitors, or DNA intercalating agents. A blockade of replication forks often results in breakage of the DNA molecules, and in the activation of an ATR/ATM dependent S-phase checkpoint pathway that senses the damage and mediates cellular responses to drug treatment. There is a continuous need for anticancer agents in order to optimise the therapeutic treatment. The anticancer research is typically focused on new agents with higher selectivity for tumor cells and lesser toxicity for the host. In particular, there is a need for new anticancer combinations capable to synergistically enhance the antitumor activity of the corresponding agents when used alone, thus allowing a substantial reduction in the amount of cytotoxic compound. In addition there is a need for new antitumor compositions, capable to display a prolonged antitumor activity, without causing a corresponding increase in toxicity for the host.

The present invention fulfils these needs by providing new combinations of a selected group of PARP-1 inhibitor with particular classes of antineoplastic agents; these combinations were found particularly suitable for the treatment of tumors. The combinations of the present invention are very useful in therapy as antitumor agents and lack, in terms of both toxicity and side effects, the drawbacks associated with currently available antitumor drugs. The combination thus provides a significant synergistic effect, as well as a prolonged tumor regression activity without correspondent increase in toxicity.

### DESCRIPTION OF THE INVENTION

The present invention provides, in a first aspect, a therapeutic combination comprising:

(a) a compound belonging to a selected group of PARP-1 inhibitors, and

(b) one or more selected antineoplastic agents.

The compound (a) is defined by the following structural formula (I):

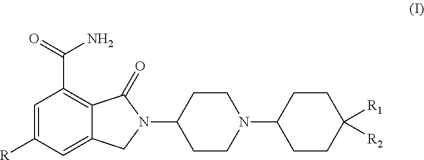

wherein R is hydrogen or halogen atom, R₁ and R₂ are both chlorine atoms, fluorine atoms or together form an oxo group (=O), and pharmaceutically acceptable salts or hydrates thereof.

In a first preferred embodiment, in formula (I), when R is hydrogen atom, then R₁ and R₂ are both fluorine atoms and, when R is fluorine atom, then R₁ and R₂ are both chlorine atoms, fluorine atoms or together form an oxo group (=O).

In a second preferred embodiment, in formula (I), R is hydrogen atom or fluorine atom, and R₁ and R₂ are both fluorine atoms.

In a third more preferred embodiment, in formula (I), R, R₁ and R₂ are all fluorine atoms.

Specific compounds of formula (I) are the following:

Compound 1:

2-[1-(4,4-difluorocyclohexyl)piperidin-4-yl]-3-oxo-2,3-dihydro-1H-isoindole-4-carboxamide;

Compound 2:

2-[1-(4,4-difluorocyclohexyl)piperidin-4-yl]-6-fluoro-3-oxo-2,3-dihydro-1H-isoindole-4-carboxamide;

Compound 3:

6-fluoro-3-oxo-2-[1-(4-oxocyclohexyl)piperidin-4-yl]-2,3-dihydro-1H-isoindole-4-carboxamide, and

Compound 4:

2-[1-(4,4-dichlorocyclohexyl)piperidin-4-yl]-6-fluoro-3-oxo-2,3-dihydro-1H-isoindole-4 carboxamide, as well as hydrates or pharmaceutically acceptable salts thereof.

These compounds and their methods of preparation have been described in the co-pending application PCT/EP2010/059607 in the name of the present Applicant.

Pharmaceutically acceptable salts of the compound of formula (I) include the acid addition salts with inorganic or organic acids, e.g., nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid and the like.

The antineoplastic agents to be used in combination with the compounds of formula (I) are those selected from the group consisting of alkylating or alkylating-like agents, antimetabolite agents, topoisomerase I inhibitors, topoisomerase II inhibitors, an antimitotic agent, and radiation.

The invention also relates to the combination of compound (a) and agents (b) described above, for use in therapy.

The invention further relates to the combination of compound (a) and agents (b) described above, for treating or delaying the progression of tumors. The invention also includes the use of the combination of compound (a) and agents (b) described above in the preparation of a medicament for treating or delaying the progression of tumors. In a further aspect the invention relates to a method of treating or delaying the progression of tumors, comprising the administration of a combination of compound (a) and agents (b) described above to a patient in need thereof. All the above combinations, uses and methods, can be performed indifferently by simultaneous, separate or sequential administration of the compound (a) and agents (b); said combinations, uses and methods can be performed by administering the compound (a) and agents (b) as compounds as such, or as pharmaceutical compositions (where the compound (a) and agents (b) can be formulated jointly or separated). When the administration is not simultaneous, the compound and the agents can be administered in any order.

The tumors to be treated, or whose progression is delayed, in accordance with the present invention include, without limitation:
carcinomas such as breast (including triple negative and BRCA mutated), ovary (including BRCA mutated), gastric, colorectal, renal, kidney, liver, lung, including small and non small cell lung cancer, esophagus, gall-bladder, bladder, pancreas, cervix, uterus, fallopian tubes, peritoneum, endometrium, thyroid, prostate (including pTEN negative), skin, including squamous cell carcinoma;
hematopoietic tumors of lymphoid lineage, including leukemia, acute lymphocitic leukemia, acute lymphoblastic leukemia, chronic lymphocitic leukemia, B-cell lymphoma, T-cell-lymphoma, Hodgkin's lymphoma, non-Hodgkin's lymphoma, hairy cell lymphoma, mantle cell lymphoma and Burkitt's lymphoma; hematopoietic tumors of myeloid lineage, including acute and chronic myelogenous leukemias, multiple myeloma, myelodysplastic syndrome and promyelocytic leukemia;
tumors of mesenchymal origin, including Ewing sarcoma, fibrosarcoma and rhabdomyosarcoma;
tumors of the central and peripheral nervous system, including astrocytoma, neuroblastoma, medulloblastoma, glioma, glioblastoma multiforme and schwannomas;
other tumors, including adenocortical cancer, melanoma, seminoma, teratocarcinoma, osteosarcoma, mesothelioma, xeroderma pigmentosum, keratoxanthoma and Kaposi's sarcoma.

In a still further aspect the invention provides a pharmaceutical composition comprising a combination of compound (a) and agents (b) described above, admixed with a pharmaceutically acceptable carrier, diluent or excipient.

According to a preferred embodiment of the invention, the alkylating or alkylating-like agent is selected from the group consisting of nitrogen mustards (mechlorethamine, cyclophosphamide, ifosfamide, melphalan and chlorambucil), aziridines (thiotepa), nitrosoureas (carmustine, lomustine, semustine), triazenes (dacarbazine and temozolomide) and platinum derivatives (cisplatin, oxaliplatin, carboplatin and satraplatin). Cisplatin can be administered, e.g., in the form as it is marketed, e.g. under the trademark CDDP®. Temozolomide can be administered, e.g., in the form as it is marketed, e.g. under the trademark TEMODAR®.

According to a more preferred embodiment of the invention, the alkylating or alkylating-like agent is one or more among carboplatin, cisplatin, temozolomide, dacarbazine.

An antimetabolite agent includes, but is not limited to, 5-fluorouracil, capecitabine, gemcitabine, pemetrexed, methotrexate and edatrexate. Capecitabine can be administered, e.g., in the form as it is marketed, e.g. under the trademark XELODA®. Gemcitabine can be administered, e.g., in the form as it is marketed, e.g. under the trademark GEMZAR®. Pemetrexed can be administered, e.g., in the form as it is marketed, e.g. under the trademark ALIMTA®.

According to a more preferred embodiment of the invention, the antimetabolite is gemcitabine.

A topoisomerase I inhibitor includes, but is not limited to, topotecan, irinotecan (CPT-11), SN-38 and 9-nitrocamptothecin. Irinotecan can be administered, e.g., in the form as it is marketed, e.g. under the trademark CAMPTOSAR®. Topotecan can be administered, e.g., in the form as it is marketed, e.g. under the trademark HYCAMTIN®.

According to a more preferred embodiment of the invention, the topoisomerase I inhibitor is one or more among irinotecan and topotecan.

A topoisomerase II inhibitor includes, but is not limited to, anthracyclines (doxorubicin, daunorubicin, epirubicin, nemorubicin and idarubicin), podophillotoxins (etoposide and teniposide), anthraquinones (mitoxanthrone and losozanthrone) and acridines (actinomycin D, bleomycin and mitomycin). Etoposide can be administered, e.g., in the form as it is marketed, e.g. under the trademark EPOSIN®.

According to a more preferred embodiment of the invention, the topoisomerase II inhibitor is nemorubicin.

An antimitotic agent includes, but it is not limited to, taxanes (paclitaxel and docetaxel). Paclitaxel can be administered, e.g., in the form as it is marketed, e.g. under the trademark TAXOL®.

According to a more preferred embodiment of the invention, the antimitotic agent is one or more among paclitaxel and docetaxel.

The term “radiation” used herein includes all antitumor treatments by means of ionizing radiation, to be preformed according to all the available techniques: a non-limitative list thereof includes: total body irradiation, fractionated radiotherapy, accelerated irradiation, intensity-modulated radiation therapy, image-guided radiation therapy, external beam radiation therapy, sealed source radiotherapy or brachytherapy, unsealed source radiotherapy, systemic radioisotope therapy, three-dimensional conformal radiotherapy, proton therapy, etc. A patient undergoing radiation therapy will be administered a physical compound (in case of radioisotopes), or not. In the latter case, the term “combination” used herein is broadly understood as the synergistic mixture of the effects provided by radiations and the compound of formula (I).

In the present invention, each of the active ingredients of the combination is in amount effective to produce a synergic antineoplastic effect.

The present invention also provides a method for lowering the side effects caused by antineoplastic therapy with an antineoplastic agent in mammals, including humans, in need thereof, the method comprising administering to said mammal a combined preparation comprising the compound of formula (I) as defined above and one or more antineoplastic agents selected from the group consisting of an alkylating or alkylating-like agent, an antimetabolite agent, a topoisomerase I inhibitor, a topoisomerase II inhibitor, an antimitotic agent, and radiation, to produce a synergic antineoplastic effect.

By the term “a synergic antineoplastic effect” as used herein is meant the inhibition of the growth tumor, or the delaying of its progression, by administering an effective amount of the combination of the compound of formula (I) as defined above and an alkylating or alkylating-like agent, an antimetabolite agent, a topoisomerase I inhibitor, a topoisomerase II inhibitor, an antimitotic agent and radiation to mammals, including human.

The term “combined preparation” as used herein also includes preparation in the form of “kit of parts” wherein the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. There is at least one beneficial effect, e.g., a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The effect of the combination of the invention is significantly increased without a parallel increased toxicity. In other words, the combined therapy of the present invention enhances the antitumoral effects of the partner (a) and/or of partner (b) of the combination of the invention and thus yields the most effective and less toxic treatment for tumors. Moreover, the combination of partners (a) and (b) provides the additional advantage of a longer tumor regression activity after administration, without this being reflected in a corresponding increase of toxicity for the organism. By the term “administered” or “administering” as used herein is meant parenteral and /or oral administration. By “parenteral” is meant intravenous, subcutaneous and intramuscolar administration.

In the method of the subject invention, for the administration of the compound of formula (I), the course of therapy generally employed is

in the range from 1 mg/m² to 1 g/m² as free base. More preferably, the course therapy employed is from about 50 mg/m²/day to about 500 mg/m²/day as free base. Typical regimens comprises the following administration schedules: daily for up to 21 consecutive days; daily for 7 consecutive days, followed by a rest period of one week for a total of 14-day cycle (two-weeks cycle); daily for 14 days, followed by a rest period of one week (three-weeks cycle); daily on days 1 to 7 and 15 to 21 of a four-weeks cycle; continuous until disease progression.

The compound of formula (I) can be administered in a variety of dosage forms, e.g., orally, in the form of tablets, capsules, sugar or film coated tablets, liquid solutions or suspensions; rectally in the form of suppositories; parenterally, e.g., intramuscularly, or through intravenous and/or intrathecal and/or intraspinal injection or infusion. In the method of the subject invention, for the administration of an alkylating agent, e.g. temozolomide, the course of therapy generally employed is from 15 mg/m² to 300 mg/m² daily. More preferably, the course of therapy generally employed is from about 50 mg/m² to 150 mg/m² daily for up to 42 consecutive days.

For the administration of a platinum derivative, e.g. cisplatin, the course of therapy generally employed is from 10 mg/m²/day to 100 mg/m²/day every 2-4 weeks. More preferably, the course of therapy generally employed is from about 50 mg/m² to 100 mg/m² on day 1, once every 3-4 weeks.

For the administration of carboplatin, the course of therapy generally employed depends on the systemic exposure (expressed as AUC value), the renal function of the patient and on the schedule of administration. A regimen targeting an AUC of from 4 to 6 mg/mL/min over a 2 to 4 week schedule is usually adopted. More preferably, a regimen targeting an AUC of 5 mg/mL/min over a 4-week schedule is used.

For the administration of an antimetabolite agent, e.g. gemcitabine or pemetrexed, the course of therapy generally employed is from 200 mg/m² to 2000 mg/m² as weekly administration. More preferably, the course of therapy generally employed is from about 500 mg/m² to 1250 mg/m² on days 1 and 8 of a 21-days cycle or on days 1, 8, 15 of a 28-day cycle (gemcitabine) or on days 1 of a 21-day cycle (pemetrexed).

For the administration of a topoisomerase I inhibitor, e.g. irinotecan, the course of therapy generally employed is from 35 mg/m² to 350 mg/m² on days 1, 8, 15, 22 of a 42-day cycle or on days 1,15, 29 of a 42-day cycle or on day 1 of a 21-day cycle. More preferably, the course of therapy generally employed is 125 mg/m² on days 1, 8, 15, 22 and 29 of a 42-day cycle.

For the administration of a topoisomerase II inhibitor, e.g. etoposide, the course of therapy generally employed is from 10 mg/m² to 200 mg/m² daily, preferably from 35 to 100 mg/m² daily, for 3 to 5 days of a 21 or 28-day cycle or on days 1, 3, 5 of a 21 or 28-day cycle. These dosages are intended for i.v. administration; in case of oral administration doses are doubled.

For the administration of an antimitotic agent, e.g. paclitaxel, the course of therapy generally employed is from 50 mg/m² to 175 mg/m² on day 1 of a 14 or 21-day cycle or from 30 mg/m² weekly. More preferably, the course of therapy generally employed is 175 mg/m² on day 1 of a 21-day cycle.

The present invention further provides a commercial package (kit of parts) comprising, in a suitable container mean, (a) a compound of formula (I) as defined above, and (b) one or more antineoplastic agents as described above, wherein the active ingredients are present in each case in free form or in the form of a pharmaceutically acceptable salt or any hydrate thereof, together with instructions for simultaneous, separate or sequential use thereof. In a package according to the invention each of partner (a) and (b) are present within a single container mean or within distinct container means.

Another embodiment of the present invention is a commercial package comprising a pharmaceutical composition or product as described above.

The activities of the combination of the present invention are shown for instance by the following in vitro and in vivo tests, which are intended to illustrate but not to limit the present invention.

### EXAMPLES

### Evaluation of in vitro Synergism

Materials and methods. Exponentially growing human cancer cell line (MDA-MB-436, MDA-MB-468, A-375, HCT-116, KM-12) was seeded and incubated at 37° C. in a humidified 5% CO₂ atmosphere. Drugs were added to the experimental culture, and incubations were carried out at 37° C. for 6 days in the dark. Serial dilution curves were prepared in medium by using a liquid handler Multiprobe II (PerkinElmer). Scalar doses of the compound of formula (I) and antineoplastic agents were added to the medium 24 hours after seeding. Drug solutions were prepared immediately before use. At the end of treatment, cell proliferation was determined by an intracellular adenosine triphosphate monitoring system (CellTiterGlo- Promega) using an Envision (PerkinElmer) reader. Inhibitory activity was evaluated comparing treated versus control data using the Assay Explorer (MDL) program. The dose inhibiting 50% of cell growth was calculated using sigmoidal interpolation curve.

In the examples 6 and 7, MDA-MB-468 cells were seeded and, 24 hours later, were treated with the PARP inhibitor and the cytotoxic drug. After addition of compound, plates are returned to the incubator for 48 hours and then were stained with a live nuclear staining Hoechst 33342 (Absorption maximum 346 nm fluorescence maximum 460 nm) (4′,6-Diamidini-2-phenyindole, dilactate) (Sigma cat. N°D 9564) a high sensitivity dye to detect nucleid acid. Percentage of cells with condensed-fragmented nuclei were calculated by using Array Scan vTi™ (Cellomics ThermoScientific) an automatic microscopy reader. The ArrayScan vTi instrument, with a Zeiss 5× 0.5 N.A. objective, and applying the Cytotoxity.V3 algorithm (Cellomics/Thermo Fisher) with a XF100 filter. At least 900 cells were read for each well. A homemade Excel macro was used to calculate percentage of cells with condensed- fragmented nuclei, in comparison with untreated samples.

Combination indices (C.I.) were calculated using a proprietary computer program for multiple drug effect analysis based on the equation of Chou-Talalay (Adv Enzyme Regul 1984;22:27-55) for mutually nonexclusive drugs, where a C.I. of <1 indicates a more than additive effect: C.I.: >3 strong antagonism; 1.3-3 antagonism; 1.2-0.8 additivity; 0.8-0.3 synergism; <0.3 strong synergism.

### Example 1

In vitro cytotoxic activity of Compound 2 of formula (I) in combination with temozolomide.

The results obtained with the drugs in combination for the MDA-MB-436 human breast cancer BRCA-1 mutated cell line are shown in Table 1.

TABLE 1
In vitro combination of Compound 2 of formula (I) with temozolomide

| | | Combination | Combination | |
|---|---|---|---|---|
| | | Index at 50% | Index at 50% | Effect of the |
| Ratio | Schedule | inhibition | inhibition | combination |
| 1:30 | simultaneous | 0.31 | 0.31 | strong synergism |
| 1:60 | simultaneous | 0.32 | 0.30 | strong synergism |
| 1:120 | simultaneous | 0.27 | 0.27 | strong synergism |
| 1:240 | simultaneous | 0.29 | 0.31 | strong synergism |

The results show that on human tumor cells Compound 2 of formula (I) can be effectively combined with the alkylating agent temozolomide producing strong synergistic effect.

### Example 2

In vitro cytotoxic activity of Compound 2 of formula (I) in combination with temozolomide.

The results obtained with the drugs in combination for the A-375 human melanoma cell line are shown in Table 2.

TABLE 2
In vitro combination of Compound 2 of formula (I) with temozolomide in A-375.

| Drug | | | | | |
|---|---|---|---|---|---|
| Compound 2 of | Temozolomide | | IC₅₀ | | |
| formula (I) μM | μM | | combination | Combination | Effect of the |
| (IC₅₀ = 32.5 μM) | (IC₅₀ = 932 μM) | Schedule | μM | Index | combination |
| 40 | 1000 | Simultaneous | 2.2 | 0.17 | Strong synergism |
| 40 | 333 | | | 0.13 | Strong synergism |
| 40 | 111 | | | 0.21 | Strong synergism |
| 20 | 1000 | Simultaneous | 52 | 0.14 | Strong synergism |
| 20 | 333 | | | 0.09 | Strong synergism |
| 20 | 111 | | | 0.22 | Strong synergism |
| 10 | 1000 | Simultaneous | 164 | 0.13 | Strong synergism |
| 10 | 333 | | | 0.14 | Strong synergism |
| 5 | 1000 | Simultaneous | 230 | 0.03 | Strong synergism |
| 5 | 333 | | | 0.25 | Strong synergism |
| 2.5 | 1000 | Simultaneous | 388 | 0.03 | Strong synergism |
| 1.25 | 1000 | Simultaneous | 379 | 0.02 | Strong synergism |
| 0.62 | 0.003 | Simultaneous | 368 | 0.02 | Strong synergism |

The results show that on human tumor cells Compound 2 of formula (I) can be effectively combined with the alkylating agent temozolomide producing strong synergistic effect in melanoma cells.

### Example 3

In vitro cytotoxic activity of Compound 2 of formula (I) in combination with cisplatin.

The results obtained with the drugs in combination for the MDA-MB-468 human breast cancer pTEN negative cell line are shown in Table 3.

TABLE 3
In vitro combination of Compound 2 of formula (I)
with cisplatin in MDA-MB-468.

| | | Combination | Combination | |
|---|---|---|---|---|
| | | index at 70% | index at 90% | Effect of the |
| Ratio | Schedule | inhibition | inhibition | combination |
| 0.02:1 | simultaneous | 0.72 | 0.56 | Synergism |
| 0.04:1 | simultaneous | 0.69 | 0.52 | Synergism |
| 0.01:1 | simultaneous | ND | 0.65 | Synergism |
| 0.005:1 | simultaneous | ND | 0.66 | Synergism |

The results show that on human tumor cells Compound 2 of formula (I) can be effectively combined with the alkylating agent cisplatin producing synergistic effect in breast cancer cells.

### Example 4

In vitro cytotoxic activity of Compound 2 of formula (I) in combination with SN-38 (the active metabolite of irinotecan).

The results obtained with the drugs in combination for the HCT-116 human colon cancer cell line are shown in Table 4.

TABLE 4
In vitro combination of Compound 2 of formula (I) with
SN-38 in HCT-116 colon cancer cells.

| Drug | | | | | |
|---|---|---|---|---|---|
| Compound | | | | | |
| 2 of formula | SN-38 μpM | | IC₅₀ | | |
| (I) μM (IC₅₀ = | (IC50 = | | combination | Combination | Effect of the |
| 14.1 μM) | 0.0011 μM) | Schedule | μM | Index | combination |
| 10 | 0.003 | Simultaneous | 0.0001 | 0.27 | Strong |
| | | | | | synergism |
| 10 | 0.001 | | | 0.19 | Strong |
| | | | | | synergism |
| 10 | 0.0003 | | | 0.47 | synergism |
| 5 | 0.003 | Simultaneous | 0.0004 | 0.19 | Strong |
| | | | | | synergism |
| 5 | 0.001 | | | 0.20 | Strong |
| | | | | | synergism |
| 5 | 0.0003 | | | 0.68 | synergism |
| 2.5 | 0.003 | Simultaneous | 0.0006 | 0.21 | Strong |
| | | | | | synergism |
| 2.5 | 0.001 | | | 0.22 | Strong |
| | | | | | synergism |
| 1.25 | 0.003 | Simultaneous | 0.0006 | 0.20 | Strong |
| | | | | | synergism |
| 1.25 | 0.001 | | | 0.22 | Strong |
| | | | | | synergism |
| 0.62 | 0.003 | Simultaneous | 0.0007 | 0.23 | Strong |
| | | | | | synergism |
| 0.62 | 0.001 | | | 0.34 | synergism |
| 0.31 | 0.003 | Simultaneous | 0.0007 | 0.26 | Strong |
| | | | | | synergism |
| 0.31 | 0.001 | | | 0.30 | Strong |
| | | | | | synergism |
| 0.16 | 0.003 | Simultaneous | 0.0009 | 0.26 | Strong |
| | | | | | synergism |
| 0.16 | 0.001 | | | 0.39 | synergism |

The results show that on human tumor cells Compound 2 of formula (I) can be effectively combined with the topoisomerase I inhibitor producing strong synergistic effect in this colon cancer cells.

### Example 5

In vitro cytotoxic activity of Compound 2 of formula (I) in combination with SN-38 (the active metabolite of irinotecan).

The results obtained with the drugs in combination for the KM-12 human colon cancer cell line are shown in Table 5.

TABLE 5
In vitro combination of Compound 2 of formula (I) with SN-38 in KM-12 colon cancer cells.

| Drug | | | | | |
|---|---|---|---|---|---|
| Compound 2 of | | | IC₅₀ | | |
| formula (I) μM | SN-38 μM | | combination | Combination | Effect of the |
| (IC₅₀ = 19.4 μM) | (IC₅₀ = 0.0015 μM) | Schedule | μM | Index | combination |
| 10 | 0.003 | Simultaneous | 0.0005 | 0.44 | synergism |
| 10 | 0.001 | | | 0.57 | synergism |
| 5 | 0.003 | Simultaneous | 0.0008 | 0.42 | synergism |
| 5 | 0.001 | | | 0.68 | synergism |
| 2.5 | 0.003 | Simultaneous | 0.0011 | 0.49 | synergism |
| 1.25 | 0.003 | Simultaneous | 0.0011 | 0.54 | synergism |
| 0.62 | 0.003 | Simultaneous | 0.0013 | 0.70 | synergism |
| 0.31 | 0.003 | Simultaneous | 0.0011 | 0.53 | synergism |
| 0.16 | 0.003 | Simultaneous | 0.0013 | 0.60 | synergism |

The results show that on human tumor cells Compound 2 of formula (I) can be effectively combined with the topoisomerase I inhibitor producing synergistic effect also in this pTEN mutated colon cancer cell line.

### Example 6

In vitro cytotoxic activity of Compound 2 of formula (I) in combination with paclitaxel.

The results obtained with the drugs in combination for the MDA-MB-468 human breast cancer cell line are shown in Table 6.

TABLE 6
In vitro combination of Compound 2 of formula (I) with paclitaxel in
MDA-MB-468 human breast pTEN mutated cancer cell line.

| | | Combination | Combination | |
|---|---|---|---|---|
| | | index | index | |
| | | at 50% | at 70% | Effect of the |
| Ratio | Schedule | inhibition | inhibition | combination |
| 3000:1 | simultaneous | 0.04 | 0.03 | Strong Synergism |
| 1000:1 | simultaneous | 0.14 | 0.12 | Strong Synergism |
| 333:1 | simultaneous | 0.02 | 0.01 | Strong Synergism |
| 111:1 | simultaneous | 0.05 | 0.02 | Strong Synergism |
| 37:1 | simultaneous | 0.07 | 0.05 | Strong Synergism |
| 12.1 | simultaneous | 0.09 | 0.07 | Strong Synergism |
| 4:1 | simultaneous | 0.32 | 0.37 | Synergism |
| 1.3:1 | simultaneous | 0.35 | 0.26 | Strong Synergism |

The results show that on human tumor cells Compound 2 of formula (I) can be effectively combined with the paclitaxel producing synergistic effect in this pTEN mutated breast cancer cell line.

### Example 7

In vitro cytotoxic activity of Compound 2 of formula (I) in combination with nemorubicin.

The results obtained with the drugs in combination for the MDA-MB-468 human breast cancer cell line are shown in Table 7.

TABLE 7
In vitro combination of Compound 2 of formula (I) with nemorubicin in
MDA-MB-468 human breast pTEN mutated cancer cell line.

| | | Combination | Combination | |
|---|---|---|---|---|
| | | index | index | |
| | | at 50% | at 70% | Effect of the |
| Ratio | Schedule | inhibition | inhibition | combination |
| 3000:1 | simultaneous | 0.34 | 0.34 | Synergism |
| 1000:1 | simultaneous | 0.47 | 0.45 | Synergism |
| 333:1 | simultaneous | 0.59 | 0.54 | Synergism |
| 111:1 | simultaneous | 0.62 | 0.53 | Synergism |
| 37:1 | simultaneous | 0.74 | 0.57 | Synergism |
| 12.1 | simultaneous | 0.76 | 0.67 | Synergism |
| 4:1 | simultaneous | 0.77 | 0.61 | Synergism |

The results show that on human tumor cells Compound 2 of formula (I) can be effectively combined with nemorubicin producing synergistic effect in this pTEN mutated breast cancer cell line.

Evaluation of Tumor Growth Inhibition and Toxicity

Materials and Methods

Balb, Nu/Nu male mice, from Harlan (Italy) or CD1 Nu/Nu female mice from CRI, were maintained in agreement with the European Communities Council Directive no. 86/609/EEC, in cages with paper filter cover, food and bedding sterilized and acidified water. Fragments of Capan-1 human pancreatic cancer tumors, MX-1 human breast cancer tumors and HCT-116 human colon carcinoma were implanted subcutaneously. KM-12 cells were grown in vitro and 3×106 in 0,2 ml were implanted subcutaneously. The treatment started when tumors were palpable.

Compounds of formula (I) were administered by oral route in a volume of 10 ml/kg at the indicated doses from day 1 to 12. Temozolomide was administered orally at the dose of 62.5 mg/kg for 5 days. When combined, compound of formula (I) was administered from day 1 to 12 and temozolomide from day 3 to day 7. Irinotecan was administered intravenous at the dose of 45 mg/kg 2 or 3 times every 4 days. When combined, compound of formula (I) was administered from day 1 to 8 or to 12 and irinotecan on day 3 and day 7 or and days 1, 5 and 9. Docetaxel was administered intravenously at the dose of 5 mg/kg once a week for three times. When combined, compound of formula (I) was administered from day 1 to 12 and docetaxel on days 1, 8 and 15.

Tumor growth and body weight were measured every 3 days. Tumor growth was assessed by caliper. The two diameters were recorded and the tumor weight was calculated according the following formula: length (mm)×width² /2. The effect of the antitumor treatment was evaluated as the delay in the onset of an exponential growth of the tumor (see for references Anticancer drugs 7:437-60, 1996). This delay (T-C value) was defined as the difference of time (in days) required for the treatment group (T) and the control group (C) tumors to reach a predetermined size (1 g). Toxicity was evaluated on the basis of number of death animals during the experiment. The results are reported in tables 6, 7, 8 and 9.

### Example 8

In vivo anti-tumor activity of Compound 1 of formula (I) in combination with temozolomide.

The results obtained with the drugs as single agents and in combination for the Capan-1 BRCA-2 mutated human pancreatic xenograft model are shown in Table 8.

TABLE 8

| Treatment | T-C (days) | Toxicity |
|---|---|---|
| Compound 1 100 mg/kg* | 5 | 0/7 |
| Temozolomide 62.5 mg/kg** | 5 | 0/7 |
| Temozolomide 62.5 mg/kg + | 48 | 0/7 |
| Compound 1 100 mg/kg*** | | |

*Oral treatments made on days 1 to 12.
** Oral treatments made on days from 3 to 7.
***Compound 1 treatments from days 1 to 12, temozolomide treatments on days from 3 to 7.

Result: The T-C observed when Compound 1 of formula (I) was combined with temozolomide was higher to the expected by the simple addition of T-C obtained by the single treatments indicating strong synergism. No toxicity was observed in any of the treatment group.

### Example 9

In vivo anti-tumor activity of Compound 2 of formula (I) in combination with temozolomide

The results obtained with the drugs as single agents and in combination for the Capan-1 human pancreatic xenograft model are shown in Table 9.

TABLE 9

| Treatment | T-C (days) | Toxicity |
|---|---|---|
| Compound 2 50 mg/kg* | 6 | 0/7 |
| Temozolomide 62.5 mg/kg** | 9 | 0/7 |
| Temozolomide 62.5 mg/kg + | 21 | 0/7 |
| Compound 2 50 mg/kg*** | | |

*Oral treatments made on days 1 to 12.
**Oral treatments made on days from 3 to 7.
***Compound 2 treatments from days 1 to 12, temozolomide treatments on days from 3 to 7.

Result: The T-C observed when Compound 2 of formula (I) was combined with temozolomide was higher to the expected by the simple addition of T-C obtained by the single treatments indicating strong synergism. No toxicity was observed in any of the treatment group.

### Example 10

In vivo anti-tumor activity of Compound 2 of formula (I) in combination with irinotecan.

The results obtained with the drugs as single agents and in combination for the KM-12 human colorectal cancer xenograft model pTEN mutated are shown in Table 10.

TABLE 10

| Treatment | T-C (days) | Toxicity |
|---|---|---|
| Compound 2 50 mg/kg* | 1 | 0/7 |
| Irinotecan 45 mg/kg** | 1 | 0/7 |
| Irinotecan 45 mg/kg + | 4 | 0/7 |
| Compound 2 50 mg/kg*** | | |

*Oral treatments made on days 1 to 12.
**Intravenous treatments made on days 3 and 7.
***Compound 2 treatments from days 1 to 12, irinotecan treatments on days 3 and 7.

Result: The T-C observed when Compound 2 of formula (I) was combined with irinotecan was higher to the expected by the simple addition of T-C obtained by the single treatments indicating strong synergism. No toxicity was observed in any of the treatment group.

### Example 11

In vivo anti-tumor activity of Compound 2 of formula (I) in combination with irinotecan.

The results obtained with the drugs as single agents and in combination for the HCT-116 human colorectal cancer xenograft model are shown in Table 11.

TABLE 11

| Treatment | T-C (days) | Toxicity |
|---|---|---|
| Compound 2 50 mg/kg* | 1 | 0/7 |
| Compound 2 100 mg/kg* | 4 | 0/7 |
| Irinotecan 45 mg/kg** | 16 | 0/7 |
| Irinotecan 45 mg/kg + | 18 | 0/7 |
| Compound 2 50 mg/kg*** | | |
| Irinotecan 45 mg/kg + | 22 | 0/7 |
| Compound 2 100 mg/kg*** | | |

*Oral treatments made on days 1 to 8.
** Intravenous treatments made on days 1, 5 and 9.
***Compound 2 treatments from days 1 to 8, irinotecan treatments on days 1, 5 and 9.

Result: The T-C observed when Compound 2 of formula (I) was combined with irinotecan was higher to the expected by the simple addition of T-C obtained by the single treatments indicating slight synergism also in this colorectal cancer model. The combination of irinotecan and the compound 2 given at the higher dose level gave a maximal tumor growth inhibition of 91%. No toxicity was observed in any of the treatment group.

### Example 12

In vivo anti-tumor activity of Compound 2 of formula (I) in combination with docetaxel.

The results obtained with the drugs as single agents and in combination for the MX-1 human breast cancer xenograft model BRCA-1 mutated are shown in Table 12.

TABLE 12

| Treatment | T-C (days) | Toxicity |
|---|---|---|
| Compound 2 100 mg/kg* | 2 | 0/7 |
| Docetaxel 5 mg/kg** | 3 | 0/7 |
| Docetaxel 5 5 mg/kg + | 16 | 0/7 |
| Compound 2 100 mg/kg*** | | |

*Oral treatments made on days 1 to 8.
**Intravenous treatments made on days 1, 8 and 15.
***Compound 2 treatments from days 1 to 12, Docetaxel treatments on days 1, 8 and 15.

Result: The T-C observed when Compound 2 of formula (I) was combined with docetaxel was higher than expected by the simple addition of T-C obtained by the single treatments indicating strong synergism in this triple negative cancer model. The combination of docetaxel and the compound 2 of formula (I) gave a maximal tumor growth inhibition of 79% and 1/7 animals was tumor free. No toxicity was observed in any of the treatment group.

1. A combination comprising:
(a) a compound defined by the structural formula (I): 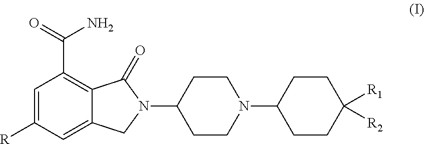
wherein R is hydrogen atom or halogen atom, R₁ and R₂ are both chlorine atoms, fluorine atoms or together form an oxo group (=O),
and pharmaceutically acceptable salts or hydrates thereof, and
(b) one or more antineoplastic agents selected from the group consisting of alkylating or alkylating-like agents, antimetabolite agents, topoisomerase I inhibitors, topoisomerase II inhibitors, an antimitotic agent and radiation.

2. A combination according to claim 1, wherein the alkylating or alkylating-like agent is selected from the group consisting of Carboplatin, Cisplatin, Temozolomide, Dacarbazine.

3. A combination according to claim 1, wherein the antimetabolite is Gemcitabine.

4. A combination according to claim 1, wherein the topoisomerase I inhibitor is selected from the group consisting of Irinotecan and Topotecan.

5. A combination according to claim 1, wherein the topoisomerase II inhibitor is nemorubicin.

6. A combination according to claim 1, wherein the antimitotic agent is selected from the group consisting of Paclitaxel and Docetaxel.

7. A combination according to claim 1 claims 1 6, wherein in formula (I):
(i): when R is hydrogen atom, then R₁ and R₂ are both fluorine atoms and, when R is fluorine atom, then R₁ and R₂ are both chlorine atoms, fluorine atoms or together form an oxo group (=O), or
(ii): R is hydrogen atom or fluorine atom, and R₁ and R₂ are both fluorine atoms, or
(iii): R, R₁ and R₂ are all fluorine atoms.

8. A combination according to claim 1 claims 1 7, wherein the compound of formula (I) is selected from the group consisting of:
2-[1-(4,4-difluorocyclohexyl)piperidin-4-yl]-3-oxo-2,3-dihydro-1H-isoindole-4-carboxamide;
2-[1-(4,4-difluorocyclohexy)piperidin-4-yl]-6-fluoro-3 -oxo-2,3-dihydro-1H-isoindole-4-carboxamide;
6-fluoro-3-oxo-2-[1-(4-oxocyclohexy)piperidin-4-yl]-2,3-dihydro-1H-isoindole-4-carboxamide, and
2-[1-(4,4-dichlorocyclohexyl)piperidin-4-yl]-6-fluoro-3-oxo-2,3-dihydro-1H-isoindole-4 carboxamide.

9. A combination according to claim 1, for simultaneous, separate or sequential administration.

10. A combination according to claim 1, formulated as a pharmaceutical composition.

11. A combination according to claim 1, in form of kit of parts comprising, in a suitable container, said agents (a) and (b), together with instructions for simultaneous, separate or sequential use thereof

12. A combination according to claim 1, for use in therapy.

13. A method for treating or delaying the progression of tumors comprising administering to a patient in need thereof a combination according to claim 1.

14. The method according to claim 13, wherein said tumors are selected from the group consisting of:
carcinomas such as breast (including triple negative and BRCA mutated), ovary (including BRCA mutated), gastric, colorectal, renal, kidney, liver, lung, including small and non small cell lung cancer, esophagus, gall-bladder, bladder, pancreas, cervix, uterus, fallopian tubes, peritoneum, endometrium, thyroid, prostate (including pTEN negative), skin, including squamous cell carcinoma;
hematopoietic tumors of lymphoid lineage, including leukemia, acute lymphocitic leukemia, acute lymphoblastic leukemia, chronic lymphocitic leukemia, B-cell lymphoma, T-cell-lymphoma, Hodgkin's lymphoma, non-Hodgkin's lymphoma, hairy cell lymphoma, mantle cell lymphoma and Burkitt's lymphoma; hematopoietic tumors of myeloid lineage, including acute and chronic myelogenous leukemias, multiple myeloma, myelodysplastic syndrome and promyelocytic leukemia;
tumors of mesenchymal origin, including Ewing sarcoma, fibrosarcoma and rhabdomyosarcoma;
tumors of the central and peripheral nervous system, including astrocytoma, neuroblastoma, medulloblastoma, glioma, glioblastoma multiforme and schwannomas;
other tumors, including adenocortical cancer, melanoma, seminoma, teratocarcinoma, osteosarcoma, mesothelioma, xeroderma pigmentosum, keratoxanthoma and Kaposi's sarcoma.

15. A method for claim 1, for use in lowering the side effects caused by antineoplastic agents selected from the group consisting of an alkylating or alkylating-like agents, antimetabolite agents, topoisomerase I inhibitors, topoisomerase II inhibitors, antimitotic agents and radiation, the said method comprising administering said antineoplastic agents in combination with a compound of formula (I) as defined in claim 1.
